(19) United States
(12) Patent Application Publication
(10) Pub. No.: US 2010/0061948 A1
(43) Pub. Date: Mar. 11, 2010

(54) COMPOSITION FOR SKIN WHITENING COMPRISING ARTEMISININE

(76) Inventors: Deok Hoon Park, Gyeonggi-do (KR); Jong Sung Lee, Gyeonggi-do (KR); Kwang Sun Jung, Gyeonggi-do (KR); Eunsun Jung, Gyeonggi-do (KR); Saebom Kim, Gyeonggi-do (KR)

Correspondence Address: JHK LAW, P.O. BOX 1078, LA CANADA, CA, 91012-1078, US

(21) Appl. No.: 12/522,381

(22) PCT Filed: Jan. 9, 2008
(86) PCT No.: PCT/KR08/00136

§ 371 Date: Jul. 7, 2009

(30) Foreign Application Priority Data
Jan. 11, 2007 (KR) 10-2007-0003238

(51) Int. Cl. A61K 8/49; C07D 493/18; A61Q 19/02

(52) U.S. Cl. 424/62; 549/279

(57) ABSTRACT

The present invention relates to a skin-whitening composition comprising artemisinine. Artemsinine according to the present invention suppresses melanin synthesis and tyrosinase activity to inhibit pigmentation, and has excellent whitening effect and safety without side effects, thereby being used for improving melasma or freckles, and whitening skin.

### TECHNICAL FIELD

The present invention relates to a skin-whitening composition comprising artemisinine.

### BACKGROUND ART

Human skin color is determined by the concentration and distribution of melanin in the skin. Melanin synthesized in the epidermal melanocytes is a polymer of polyphenols existing in the form of complexes of dark pigment and protein. It is re-sponsible for protecting the skin against UV radiation. It is known that tyrosinase present in melanocytes is the greatest contributor in melanin biosynthesis. Tyrosinase is a key enzyme in skin pigmentation, and catalyzes the conversion of tyrosine to DOPA (dihydroxyphenylalanine) and dopaquinone, which are intermediate products formed during melanin biosynthesis.

However, overproduction of the melanin may induce discoloration, melasma, freckles or the like. Accordingly, the inhibition of melanin overproduction improves skin hyperpigmentation such as melasma and freckles due to UV, hormone, and hereditary factors, as well as skin brightening and whitening effects.

In general, conventional materials having tyrosinase inhibiting activities such as hydroquinone, ascorbic acid, kojic acid, glutathione and cysteine have been used for improving skin whitening effect or hyperpigmentation. However, hydroquinone has problems in that only an extremely restricted amount should be used due to the severe skin irritation, even though it exhibits skin whitening effects. Ascorbic acid is easily oxidized, so that cosmetics blended with it have problems of color and odor changes, and kojic acid is unstable in a solution, which requires a complicated preparation process. In addition, thiol based compounds such as glutathione and cysteine have unique unpleasant odors as well as problems in transdermal absorption, and glycosides and derivatives thereof have problems in that they cannot be appropriately used as mixed ingredients of cosmetics due to their high polarities. Vitamin C is disadvantageous in that it is easily oxidized in an aqueous solution, so as not to continuously exhibit its effect.

In recent years, skin-whitening compositions containing extracts of natural medicinal herbs have been developed. However, since most of them are colored, there are limitations in blending. Further, since their effective ingredients are not identified, consistent effects cannot be expected in products.

Meanwhile, artemisinine is a substance derived from Artemisia annua L, and acts to inhibit cell wall synthesis of a malaria parasite, plasmodium berghei. Artemisinine has been known to be useful for pharmaceutical applications, such as tertian fever, chloroquine-resistant malaria, and distoma. Further, artemisinine has been used for the treatment of influenza fever, malaria, dyspepsia, stomach aches, heatstroke, dysentery, and severe furuncle in oriental medicine. Many studies have been made on the efficacy of artemisinine. However, there is no study on its skin whitening effect.

### DISCLOSURE OF INVENTION

### Technical Problem

The present inventors have made an effort to develop a skin-whitening composition having an excellent whitening effect without side effects. They found that artemisinine suppresses melanin synthesis and tyrosinase activity to inhibit pigmentation, and has excellent whitening effect and safety without side effects, thereby completing the present invention.

### Technical Solution

It is an object of the present invention to provide a cosmetic composition for whitening skin, comprising artemisinine.

Further, it is another object of the present invention to provide a pharmaceutical composition for whitening skin, comprising artemisinine.

### BEST MODE FOR CARRYING OUT THE INVENTION

The present invention provides a skin-whitening composition, comprising artemisinine represented by the following Formula 1.

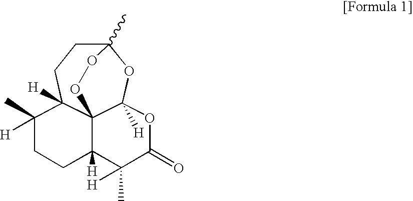

The skin-whitening composition according to the present invention comprises a cosmetic and pharmaceutical composition.

Hereinafter, the present invention will be described in detail.

Artemisinine used as an effective ingredient in the composition of the present invention may be extracted and isolated from a natural medicinal herb, for example, Artemisia annua L, or synthesized by chemical methods. Further, a commercially available artemisinine may be used.

Artemisinine according to the present invention suppresses melanin synthesis and tyrosinase activity to inhibit pigmentation, and has excellent whitening effect and safety without side effects, thereby being used for improving melasma or freckles, and whitening skin. Accordingly, the composition comprising artemisinine according to the present invention can be used in cosmetics or medicines for whitening skin.

In the skin-whitening composition of the present invention, the content of artemisinine may be 0.0001 to 15 wt %, and preferably 0.001 to 5 wt %, based on the total weight of the composition. If the content is less than 0.0001 wt %, the whitening effect is not sufficient, and if the content is more than 15 wt %, the whitening effect is slightly increased, whereas there is a problem in the stability upon formulation.

The skin-whitening composition of the present invention may include at least one known effective ingredient having skin-whitening effects, in addition to artemisinine.

To improve skin-whitening effect, the composition of the present invention may include additives commonly used in cosmetic formulations, for example, conventional auxiliary agents such as an antioxidant, a stabilizer, a solubilizing agent, a vitamin, a pigment, and a scent, and/or a carrier, in addition to artemisinine as an effective ingredient. Further, the cosmetic composition may further include a skin absorption enhancer to improve skin-whitening effect.

The cosmetic composition of the present invention can be prepared as any formulation commonly prepared in the art. The cosmetic composition can be formulated as, for example, a solution, a suspension, an emulsion, a paste, a gel, a cream, a lotion, a powder, a soap, a surfactant-containing cleanser, an oil, a powdered foundation, an emulsion foundation, a wax foundation, a spray, or the like, but not limited thereto. More specifically, the cosmetic composition can be prepared as a formulation such as a softening toner, a nutrient toner, a nutrient cream, a massage cream, an essence, an eye cream, a cleansing cream, a cleansing foam, a cleansing water, a pack, a spray, and a powder.

If the formulation of the cosmetic composition of the present invention is a paste, a cream or a gel, an animal oil, a vegetable oil, a wax, paraffin, a starch, traganth, a cellulose derivative, a polyethylene glycol, silicone, bentonite, silica, talc, zinc oxide, or the like can be used as the carrier ingredient.

If the formulation of the cosmetic composition of the present invention is a powder or a spray, lactose, talc, silica, aluminum hydroxide, calcium silicate, or polyamide powders can be used as the carrier ingredient, and in particular, if the formulation is a spray, a propellent such as chlorofluorohydrocarbon, propane/butane and dimethyl ether can be used.

If the formulation of the cosmetic composition of the present invention is a solution or an emulsion, a solvent, a solubilizing agent or an emulsifier can be used as the carrier ingredient, and examples thereof include water, ethanol, isopropanol, ethyl carbonate, ethyl acetate, benzyl alcohol, benzyl benzoate, propylene glycol, 1,3-butylene glycol oil, glycerol aliphatic esters, polyethylene glycol or sorbitan fatty acid esters.

If the formulation of the cosmetic composition of the present invention is a suspension, a liquid diluent such as water, ethanol and propylene glycol; a suspending agent such as ethoxylated isostearyl alcohol, polyoxyethylene sorbitol ester and polyoxyethylene sorbitan ester; microcrystalline cellulose; aluminum metahydroxide; bentonite; agar, traganth, or the like can be used as the carrier ingredient.

If the formulation of the cosmetic composition of the present invention is a surfactant-containing cleanser, aliphatic alcohol sulfate, aliphatic alcohol ether sulfate, sulphosuccinic acid monoester, isethionate, imidazolinium derivative, methyltaurate, sarcosinate, fatty acid amide ether sulfate, alkylamidobetain, aliphatic alcohol, fatty acid glyceride, fatty acid diethanolamide, vegetable oils, a lanolin derivative or ethoxylated glycerol fatty acid ester, or the like can be used as the carrier ingredient.

In the cosmetic composition of the present invention, the content of artemisinine may be 0.0001 to 15 wt %, and preferably 0.001 to 5 wt %, based on the total weight of the cosmetic composition.

Further, to improve skin-whitening effect, the pharmaceutical composition of the present invention can be prepared by including at least one pharmaceutically acceptable carrier, in addition to artemisinine as an effective ingredient. Examples of the pharmaceutically acceptable carrier include a saline solution, sterile water, a Ringer's solution, a buffered saline solution, a dextrose solution, a maltodextrin solution, glycerol, ethanol and a mixture of one or more thereof. If necessary, the composition may also include other conventional additives such as antioxidants, buffers, and bacteriostatic agents. Moreover, the composition may additionally include diluents, dispersants, surfactants, binders, and lubricants to formulate it into any suitable formulation including oral formulations such as powder, granule, tablet, capsule, suspension, emulsion, syrup and aerosol; external preparations such as ointment and cream; suppository, and sterilized solution for injection. Furthermore, the composition may be preferably formulated depending on particular diseases and its components, using a suitable method in the relevant field of art or the method described in Remington's Pharmaceutical Science (latest edition), Mack Publishing Company, Easton Pa.

The pharmaceutical composition of the present invention may be administered by oral or parenteral route (e.g., intravenous, subcutaneous, intraperitoneal, or topical administration) depending on its purpose. An effective dosage of the present composition may be determined depending on the patient's age, body weight, gender, health state, and diet, administration time, administration routes, excretion rates, and severity of the diseases. For topical administration, artemisinine may be preferably administered at a daily dosage of 1.0 to 3.0 ml one time or five times for 1 month or more. Further, for oral administration, artemisinine may be preferably administered at a daily dosage of 0.1 to 100 mg/kg one time or five times.

[34]

### MODE FOR THE INVENTION

Hereinafter, the preferred Examples are provided for better understanding. However, these Examples are for the illustrative purpose only, and the invention is not intended to be limited by these Examples.

### Example 1

### Inhibitory Effect on Melanin Synthesis

In order to confirm the inhibitory effect of artemisinine on melanin synthesis, murine B16 melanoma cells were used to perform the following experiment.

Artemisinine used in the present experiment was purchased from Sigma.

Murine melanoma (B16 F10) cells were inoculated on 6 well plates containing DMEM media supplemented with 10% FBS (fetal bovine serum) (1×10⁵ per well), and cultured in 5% CO₂ at37° C. until reaching about 80% confluency. Then, media was removed from the cells, replaced with fresh media, and cultured in 5% CO₂ at 37° C. for 3 days. The treatment concentration of artemisinine is determined over a range of 10 μM, 50 μM, and 100 μM, which show no cytotoxicity. Media was removed from the cells, and the cells were washed with PBS (phosphate buffered saline), followed by trypsin treatment. The cells were recovered, and counted using a hemocytometer. Then, the cells were centrifuged at 5,000 to 10,000 rpm for 10 min, and the supernatant was removed to obtain a pellet. The pellet was dried at 60° C., and suspended in 100 of 1 M sodium hydroxide solution containing 10% DMSO in a 60° C. water bath to obtain melanin in the cells. Absorbance was determined at 490 nm using a microplate reader to assess the melanin content per cell. As a control group, a known inhibitor of melanin synthesis, arbutin, was used.

The result is shown in Table 1.

TABLE 1

| | Treatment | Inhibition rate of melanin |
|---|---|---|
| Sample | concentration (μM) | synthesis (%) |
| Artemisinine | 10 | 22 |
| | 50 | 50 |
| | 100 | 65 |
| Arbutin | 100 | 52 |

As shown Table 1, artemisinine according to the present invention exhibited higher inhibition rate of melanin synthesis than arbutin.

### Example 2

### Inhibitory Effect on Tyrosinase Activity

In order to confirm the inhibitory effect of artemisinine on tyrosinase activity, murine B16 melanoma cells were used to perform the following experiment.

Murine melanoma (B16 F10) cells were inoculated on 6 well plates containing DMEM media supplemented with 10% FBS (1×10⁵ per well), and cultured in 5% CO₂ at 37° C. until reaching about 80% confluency. Then, media was removed from the cells, replaced with fresh media, and cultured in 5% CO₂ at37° C. for 3 days. The treatment concentration of artemisinine is determined over a range of 10 μM, 50 μM, and 100 μM, which show no cytotoxicity. Media was removed from the cells, and the cells were washed with PBS, followed by trypsin treatment. The cells were recovered, and counted using a hematocytometer. Then, the cells were centrifuged at 5,000 to 10,000 rpm for 10 min, and the supernatant was removed to obtain a pellet. The pellet was suspended in a lysis buffer, and centrifuged at 12,000 rpm for 10 min to collect the supernatant. Absorbance was determined at 492 nm using a microplate reader to assess the tyrosinase activity per cell. As a control group, arbutin was used.

The result is shown in Table 2.

TABLE 2

| | Treatment | Inhibition rate of tyrosinase |
|---|---|---|
| Sample | concentration (μM) | activity (%) |
| Artemisinine | 10 | 19 |
| | 50 | 38 |
| | 100 | 67 |
| Arbutin | 100 | 45 |

As shown Table 2, artemisinine according to the present invention exhibited higher inhibition rate of tyrosinase activity than arbutin.

### Example 3

### Evaluation of Whitening Effect in Animal

In order to confirm the whitening effect of artemisinine in animals, brown guinea pigs (Tortoiseshell guinea pigs), which are known to increase pigmentation upon exposure to UV like human, were used to perform the following experiment.

To cause pigmentation in the brown guinea pig by UV, light-shielding aluminum foil with windows of 3×3 cm² was adhered to hair-removed abdominal skin of brown guinea pig, and then UV light was irradiated thereon with a SE lamp (wavelength 290-320 nm, Toshiba) (total irradiation energy=1350 mJ/cm²). After UV irradiation, the aluminum foil was removed, and then the samples (artemisinine and arbutin) were applied as follows. Increased pigmentation was observed at 2 or 3 days after UV irradiation, and reached a maximum after about 2 weeks. From the maximum, each sample was applied. Applications were performed once or twice a day for 50 days. The samples were dissolved and diluted in a certain solvent (a mixed solvent of propylene glycol:ethanol:water=5:3:2) and applied with a swab. Another region was applied with the solvent only as a control. Occurrence of cumulative irritation also was examined.

The degree of skin pigmentation was determined using a chromameter (MINOLTA CR2002) to evaluate the effects. L*A*B* colorimetric system is used to classify color, and an L* value was used as standard in the present invention. The L* value was corrected using white board standard, and measured more than five times at one region, repeatedly. Pigmentation was evenly distributed. Skin color differences (ΔL*) between initial application point and terminal application point were obtained by using the following Mathematical Equation 1, and then using these values, their effects of the applied samples were evaluated.

ΔL*=L*value at terminal application day−L*value at initial application day [Mathematical Equation 1]

ΔL* values were obtained at sample application region and control application region and compared. From the result, the whitening effects of the samples can be evaluated.

The result is shown in Table 3.

TABLE 3

| | Treatment | |
|---|---|---|
| Sample | concentration (%) | Whitening effect (ΔL*) |
| Artemisinine | 0.2 | 0.40 |
| | 1.0 | 0.65 |
| Arbutin | 1.0 | 0.55 |

As shown Table 3, artemisinine according to the present invention exhibited better whitening effect than arbutin. Further, in the cumulative irritation test, artemisinine causes no cumulative irritation. Therefore, artemisinine according to the present invention was found to be safe for skin.

### Example 4

### Safety Test on Human Skin

In order to confirm the safety of artemisinine on human skin, the following experiment was performed.

1. Preparation for External Application

Skin external preparation containing artemisinine was prepared using the ingredients shown in Table 4 below. As a control group, a skin external preparation was prepared without a tyrosinase inhibitor. As an experimental group, a skin external preparation containing artemisinine was prepared, and as a comparative experimental group, a skin external preparation containing arbutin was prepared.

To prepare the skin external preparation, purified water, glycerin, and butylene glycol were mixed and dissolved at 70° C. (aqueous phase). The remaining components except for the above three components and trimethanolamine were dissolved at 70° C. (oil phase). The oil phase was added to the aqueous phase, and stirred with a Homomixer (Tolcushu Kilca company, Japan) to primarily emulsify. Finally, trimethanolamine was added thereto. Foam produced in the mixed solution was removed, and then cooled to room temperature to prepare skin external preparations.

TABLE 4

| | Content (%) | | |
|---|---|---|---|
| | Control | Experimental | Comparative |
| Ingredient | group | group | Experimental group |
| Purified water | 72.0 | 72.0 | 72.0 |
| Glycerin | 8.0 | 8.0 | 8.0 |
| Butylene glycol | 4.0 | 4.0 | 4.0 |
| Artemisinine | — | 1.0 | — |
| Arbutin | — | — | 1.0 |
| Caprylic/capric triglyceride | 8.0 | 8.0 | 8.0 |
| Squalane | 5.0 | 5.0 | 5.0 |
| Cetearyl glucoside | 1.5 | 1.5 | 1.5 |
| Sorbitan stearate | 0.4 | 0.4 | 0.4 |
| Cetearyl alcohol | 1.0 | 1.0 | 1.0 |
| Trimethanolamine | 0.1 | 0.1 | 0.1 |
| Total weight | 100 | 100 | 100 |

2. Cumulative Irritation Test

Each skin external preparation prepared in the above 1 was applied every other day to the forearms of 30 healthy adults (age: 20-40, female-15, male-15), and left for 24 hours, and this was repeated so that each subject was treated with 9 fresh patches in total, so as to confirm whether skin irritation is caused by artemisinine.

The patch test was performed using a Finn chamber (Epitest Ltd, Finland). The external preparations prepared in the above 1 were loaded dropwise in an amount of 15 per patch on the chamber. At every round of the patch application, the response of the skin was scored using the following Mathematical Equation 2.

Average response degree =[[Response index×Response degree/Total number of subjects×Highest score (4 points)]×100]÷Times of examination (9 rounds) [Mathematical Equation 2]

In regard to the response degree, 1 point was provided for ±, 2 points for +, 4 points for ++. When the average response degree was less than 3, the composition was determined to be safe for the skin.

The result is shown in Table 5.

TABLE 5

| | Nos. of Subjects in response | | | | | | | | | |
|---|---|---|---|---|---|---|---|---|---|---|
| | Week 1 | | | Week 2 | | | Week 3 | | | |
| | 1^{st} | 2^{nd} | 3^{rd} | 4^{th} | 5^{th} | 6^{th} | 7^{th} | 8^{th} | 9^{th} | Avg. Response |
| Test Material | ±+++ | ±+++ | ±+++ | ±+++ | ±+++ | ±+++ | ±+++ | ±+++ | ±+++ | Degree |
| Control group | 2−− | −−− | −−− | −−− | −−− | −−− | −−− | −−− | −−− | 0.18 |
| Experimental | 2−− | −−− | −−− | −−− | −−− | −−− | −−− | −−− | −−− | 0.18 |
| group | | | | | | | | | | |
| Comparative Experimental | 2−− | −−− | −−− | −−− | −−− | −−− | −−− | −−− | −−− | 0.18 |
| group | | | | | | | | | | |
| No. of subjects | 30 | 30 | 30 | 30 | 30 | 30 | 30 | 30 | 30 | |

As shown in Table 5, in the external preparation containing artemisinine (Experimental group), the subjects corresponding to ±, +and ++numbered 2, 0 and 0, respectively, while the others showed no response. Further, the average response degree was calculated to be 0.18, which is less than 3, demonstrating that the composition of the present invention is safe for human skin.

The external preparation containing artemisinine of the present invention (Experimental group) causes no noticeable cumulative irritation, like the control group and the external preparation containing arbutin (Comparative Experimental group). Therefore, artemisinine according to the present invention was found to be safe for human skin.

Formulation Examples for the composition of the present invention will be described in below.

### Formulation Example 1

### Preparation of Cosmetic Material

1. Softening toner (content: wt %)

Artemisinine 0.01

Glycerin 3.0

Butylene glycol 2.0

Propylene glycol 2.0

Carboxyvinylpolymer 0.1

Ethanol 10.0

Triethanolamine 0.1

Preservative, pigment, flavor, purified water residual amount

Total 100.0

2. Nutrient toner (content: wt %)

Artemisinine 0.01

Wax 4.0

Polysorbate 60 1.5

Sorbitan sesquioleate 0.5

Liquid paraffin 5.0

Squalane 5.0

Caprylic/capric triglyceride 5.0

Glycerin 3.0

Butylene glycol 3.0

Propylene glycol 3.0

Carboxyvinylpolymer 0.1

Triethanolamine 0.2

Preservative, pigment, flavor, purified water residual amount

Total 100.0

3. Nutrient cream (content: wt %)

Artemisinine 0.005

Wax 10.0

Polysorbate 60 1.5

Sorbitan sesquioleate 0.5

Liquid paraffin 10.0

Squalane 5.0

Caprylic/capric triglyceride 5.0

Glycerin 5.0

Butylene glycol 3.0

Propylene glycol 3.0

Triethanolamine 0.2

Preservative, pigment, flavor, purified water residual amount

Total 100.0

4. Massage cream (content: wt %)

Artemisinine 0.005

Wax 10.0

Polysorbate 60 1.5

Sorbitan sesquioleate 0.8

Liquid paraffin 40.0

Squalane 5.0

Caprylic/capric triglyceride 4.0

Glycerin 5.0

Butylene glycol 3.0

Propylene glycol 3.0

Triethanolamine 0.2

Preservative, pigment, flavor, purified water residual amount

Total 100.0

5. Pack (content: wt %)

Artemisinine 0.005

Polyvinylalcohol 13.0

Sodiumcarboxymethylcellulose 0.2

Allantoin 0.1

Ethanol 5.0

Nonylphenyl ether 0.3

Preservative, pigment, flavor, purified water residual amount

Total 100.0

### Formulation Example 2

### Pharmaceutical Preparation

1. Preparation of Powder Formulation

Artemisinine 2 g

Lactose 1 g

The above ingredients were mixed, and charged in an air-tight package to prepare a powder formulation.

2. Preparation of Tablet Formulation

Artemisinine 100 mg

Corn starch 100 mg

Lactose 100 mg

Magnesium stearate 2 mg

The above ingredients were mixed, and then tabletted according to a conventional preparation method to prepare a tablet formulation.

3. Preparation of Capsule Formulation

Artemisinine 100 mg

Corn starch 100 mg

Lactose 100 mg

Magnesium stearate 2 mg

The above ingredients were mixed, and then sealed in a gelatin capsule according to a conventional preparation method to prepare a capsule formulation.

### INDUSTRIAL APPLICABILITY

Artemisinine according to the present invention suppresses melanin synthesis and tyrosinase activity to inhibit pigmentation, and has excellent whitening effect and safety without side effects, thereby being used for improving melasma or freckles, and whitening skin.

1. A cosmetic composition for whitening skin, comprising artemisinine represented by the following Formula 1.

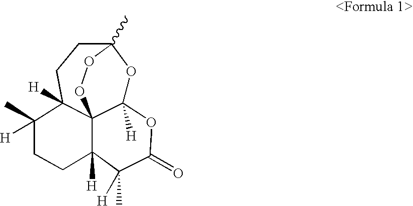

2. The cosmetic composition for whitening skin according to claim 1, wherein the content of artemisinine is 0.0001 to 15 wt %, based on the total weight of the composition.

3. A pharmaceutical composition for whitening skin, comprising artemisinine represented by the following Formula 1.

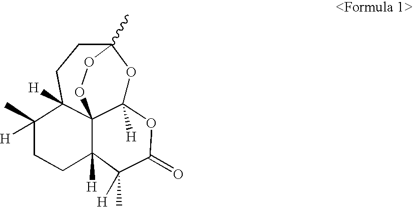

4. The pharmaceutical composition for whitening skin according to claim 3, wherein the content of artemisinine is 0.0001 to 15 wt %, based on the total weight of the composition.
